(19) United States
(12) Patent Application Publication
(10) Pub. No.: US 2005/0159701 A1
(43) Pub. Date: Jul. 21, 2005

(54) Breast pump pressure regulation valve

(76) Inventor: Jonathan W. Conaway, Hamilton, OH (US)

Correspondence Address: TAFT, STETTINIUS & HOLLISTER LLP, SUITE 1800, 425 WALNUT STREET, CINCINNATI, OH, 45202-3957, US

(21) Appl. No.: 10/758,585

(22) Filed: Jan. 15, 2004

(51) Int. Cl. A61M 1/06

(52) U.S. Cl. 604/74

(57) ABSTRACT

A pressure regulation device for selectively regulating the flow of a fluid between a lower pressure sink of a breast pump and a higher pressure source, the pressure regulation device comprising: (a) a valve body oriented with respect to a valve seat to selectively provide fluid communication between a lower pressure sink of a breast pump and a higher pressure source; and (b) an actuator operatively coupled to at least one of the valve body and the valve seat to manipulate the orientation of the valve seat with respect to the valve body, where the actuator is repositionable to selectively manipulate a volumetric flow of fluid between the higher pressure source and the lower pressure sink by varying the proximity of the valve seat with respect to the valve body.

### BACKGROUND

1. Field of the Invention

The present invention is directed to a pressure regulation device; and, more particularly, to a valve for manipulating the pressure between a low pressure sink of a breast pump and a breast.

2. Background of the Invention

Breast milk pumps are well known in the art and generally comprise a hood body or breast shield that fits over the breast, a pump connected to the hood body for generating an intermittent reduced pressure within the hood body, and a receptacle for the secreted milk.

A design consideration in manually driven breast pumps may include the pressure differential created. As individual's anatomies and discomfort levels vary, a specific pressure differential may not suit the comforts of all users. Therefore, consideration has been given to providing a pressure regulator to vary the pressure differential generated while using the pump. By manipulation of a valve, a user may accommodate her specific preferences and vary the pressure differential created without permanently reconfiguring the breast pump.

### SUMMARY OF THE INVENTION

The present invention is directed to a pressure regulation device; and, more particularly, to a valve for manipulating the pressure between a low pressure sink of a breast pump and a breast.

In an exemplary embodiment, a breast pump incorporates a bleed valve to vary the pressure differential created by the stroke of a piston within a chamber of a breast pump and the breast to which the pump is mounted. The bleed valve comprises a dial adapted to be manipulated in a clockwise or counterclockwise direction to reposition a needle interfacing an opening of a passage providing selective fluid communication between an external environment and the pressurized chamber of the breast pump. By repositioning the dial, the clearance between the needle and opening is changed, thereby changing the rate at which fluid from the external environment passes into the passage and into the pressurized chamber, effectively reducing the pressure differential between the pressure within the chamber and the ambient pressure upon the breast. The bias associated with the needle maintains the relative position of the needle with respect to the opening over a relevant use period until changed by rotating the dial.

It is a first aspect of the present invention to provide a pressure regulation device for selectively regulating the flow of a fluid between a lower pressure sink of a breast pump and a higher pressure source, the pressure regulation device comprising: (a) a valve body oriented with respect to a valve seat to selectively provide fluid communication between a lower pressure sink of a breast pump and a higher pressure source; and (b) an actuator operatively coupled to at least one of the valve body and the valve seat to manipulate the orientation of the valve seat with respect to the valve body, where the actuator is repositionable to selectively manipulate a volumetric flow of fluid between the higher pressure source and the lower pressure sink by varying the proximity of the valve seat with respect to the valve body.

It is a second aspect of the present invention to provide a pressure regulation device for selectively regulating the flow of a fluid between a lower pressure sink of a breast pump and a higher pressure source, the pressure regulation device comprising: (a) a valve body oriented and biased with respect to a valve seat to selectively provide fluid communication between a lower pressure sink of a breast pump and a higher pressure source; and (b) an actuator operatively coupled to at least one of the valve body and the valve seat to manipulate the orientation of the valve seat with respect to the valve body, where the actuator is repositionable to selectively manipulate a volumetric flow of fluid between the higher pressure source and the lower pressure sink by varying the bias of the valve body with respect to the valve seat.

It is a third aspect of the present invention to provide a pressure regulation device for selectively regulating the flow of a fluid between a lower pressure sink of a breast pump and a higher pressure source, the pressure regulation device comprising: (a) a valve body oriented with respect to a valve seat to selectively provide fluid communication between a lower pressure sink of a breast pump and a higher pressure source; and (b) an actuator operatively coupled to at least one of the valve body and the valve seat to manipulate the orientation of the valve seat with respect to the valve body, where the actuator transforms rotational movement into linear movement of at least one of the valve body and the valve seat to reposition the valve body with respect to the valve seat.

It is a fourth aspect of the present invention to provide a breast pump comprising: (a) an interface adapted to create a fluidic seal between a circumferential portion of the interface and a breast; (b) a reservoir in fluid communication with the interface for receiving milk drawn from the breast and passing by the interface; (c) a sink in fluid communication with the interface, where the sink induces a reduced pressure approximate the interface to draw milk from the breast and past the interface and into the reservoir; and (d) a pressure regulator in fluid communication with the sink to regulate the reduced pressure approximate the interface, where the pressure regulator includes a valve seat and a valve body that are selectively repositionable to manipulate the reduced pressure approximate the interface by varying the proximity of the valve seat with respect to the valve body.

It is a fifth aspect of the present invention to provide a pressure regulation device for selectively regulating the flow of a fluid between a lower pressure sink within a breast pump and a higher pressure source, the pressure regulation device comprising: (a) a valve plug circumferentially bounded, at least in part, by a housing adapted to contact a helical body to bias the valve plug with respect to a valve seat where the position of the helical body with respect to the housing determines at least in part the position of the valve plug with respect to the valve seat; and (b) an actuator operatively coupled to the helical body to manipulate the position of the valve plug with respect to the valve seat, thereby selectively providing fluid communication between a lower pressure sink of a breast pump and a higher pressure source.

It is a sixth aspect of the present invention to provide a pressure regulation device for selectively regulating the flow of a fluid between a lower pressure sink within a breast pump and a higher pressure source, the pressure regulation device comprising a dial actuator including an appendage coupled to a valve body, at least one of the appendage and the valve body having a helical body mounted thereto, the helical body biasing the dial actuator, where rotation of the dial actuator varies the position of the valve body with respect to a valve seat, having an orifice therein, for selectively regulating a volumetric flow of a fluid between a lower pressure sink within a breast pump and a higher pressure source.

### BRIEF DESCRIPTION OF THE DRAWINGS

FIG. 1 is an exploded perspective view from the front of an exemplary breast pump in accordance with the present invention;

FIG. 2 is a cross-sectional view of the exemplary breast pump in accordance with the present invention;

FIG. 3 is an exploded view of an exemplary bleed valve in accordance with the present invention;

FIG. 4 is a cross-sectional view of the exemplary bleed valve in accordance with the present invention;

FIG. 5 is an underneath view of an exemplary component of the exemplary bleed valve in accordance with the present invention;

FIG. 6 is a cross-sectional view of the exemplary component of FIG. 5 along lines A-A;

FIG. 7 is a cross-sectional view of the exemplary component of FIG. 5 along lines C-C;

FIG. 8 is an overhead view of another exemplary component of the exemplary bleed valve in accordance with the present invention;

FIG. 9 is a side view of the exemplary component of FIG. 8;

FIG. 10 is an underneath view of the exemplary component of FIG. 8;

FIG. 11 is a cross-sectional view of the exemplary component of FIG. 10, along lines A-A.

FIG. 12 is an underneath view of still a further exemplary component of the exemplary bleed valve of the present invention; and

FIG. 13 is a cross-sectional view of the exemplary component of FIG. 12, along lines A-A.

### DETAILED DESCRIPTION

The exemplary embodiments of the present invention are described and illustrated below as a pressure regulation device, and more specifically to a breast pump including a pressure regulation device operable to selectively manipulate the negative pressure created within a pressure chamber of the breast pump. The various orientational, positional, and reference terms used to describe the elements of the exemplary breast pump and pressure regulation device are therefore used according to this frame of reference. However, for clarity and precision, only a single orientational or positional reference will be utilized. Therefore, it will be understood that the positional and orientational terms used to describe the elements of the exemplary embodiments of the present invention are only used to describe the elements in relation to one another.

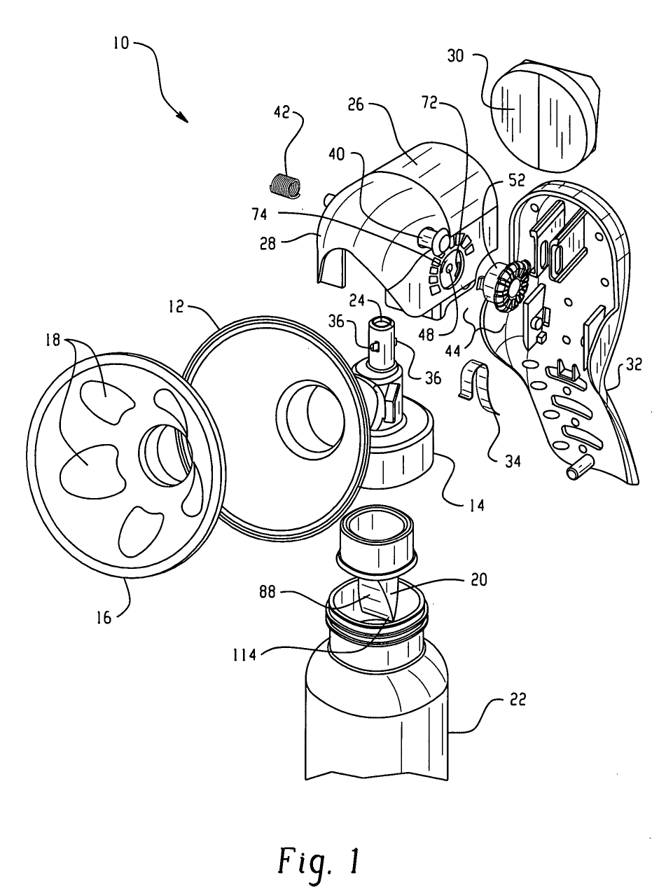

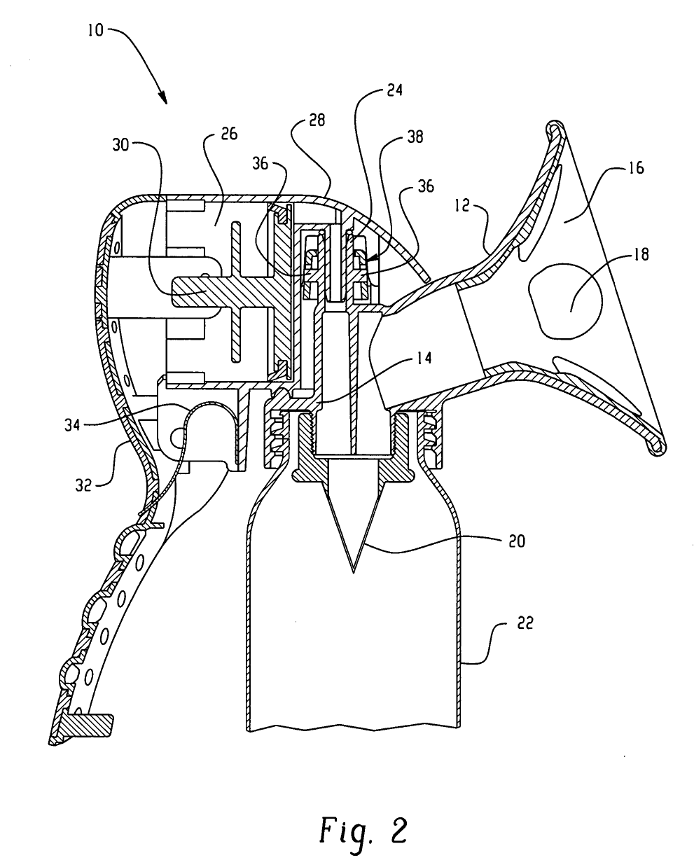

Referencing FIGS. 1 and 2, a breast pump 10 in accordance with the present invention includes a funnel 12 in sealed fluid communication with a diversion tunnel 14 for carrying away milk withdrawn from a breast. The funnel 12 includes a liner 16. The diversion tunnel 14 includes a check valve 20 in series therewith for depositing the milk withdrawn from the breast into a container 22 detachable from the diversion tunnel 14. The diversion tunnel 14 also includes a vent 24 in fluid communication with a depressurized chamber 26 occupying at least part of a housing 28. The depressurized chamber 26 includes a piston 30 riding therein that is manipulated by a handle 32 coupled thereto. A leaf spring 34 biases the handle 32 away from the housing 28 and positions the piston 30 approximate the front face of the depressurized chamber 26 in a default position.

A pair of bosses 36 axially distributed about the vent 24 engage L-shaped grooves 38 within a release pin 40 to mount the housing 28 to the vent 24 and the diversion tunnel 14. The release pin 40 is biased in the locked position by a spring 42. To disengage the housing 28 from the vent 24, the release pin 40 is pushed inward to vertically align vertical segments of the L-shaped grooves 38 with the bosses 36 that allow for vertical disengagement of the bosses 36 and grooves 38. Engagement of the housing 28 to the vent 24 is accomplished by simply vertically inserting the bosses 36 against a tapered lip (not shown) of the L-shaped grooves 38 to reposition the pin 40 horizontally and align the bosses 36 with the vertical segment of the L-shaped grooves 38 and allowing the insertion bosses 36 therein. Upon the bosses passing the vertical segment of the grooves 38, the bias associated with the pin 40 locks the bosses 36 within the horizontal segment of the L-shaped grooves 38.

A bleed valve 44 is located approximate the release pin 40 and is adapted to manipulate the volumetric flow of fluid traveling between an external environment and the depressurized chamber 26 by way of a passage (not shown). The end of the passage includes an opening 48 adapted to receive a needle 50 of the bleed valve 44.

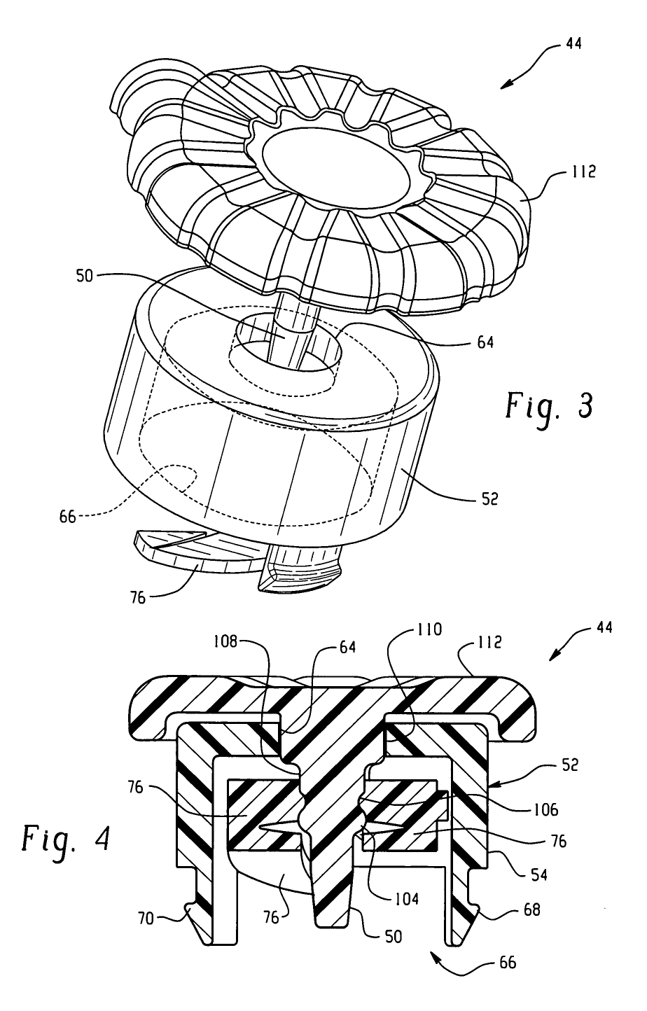

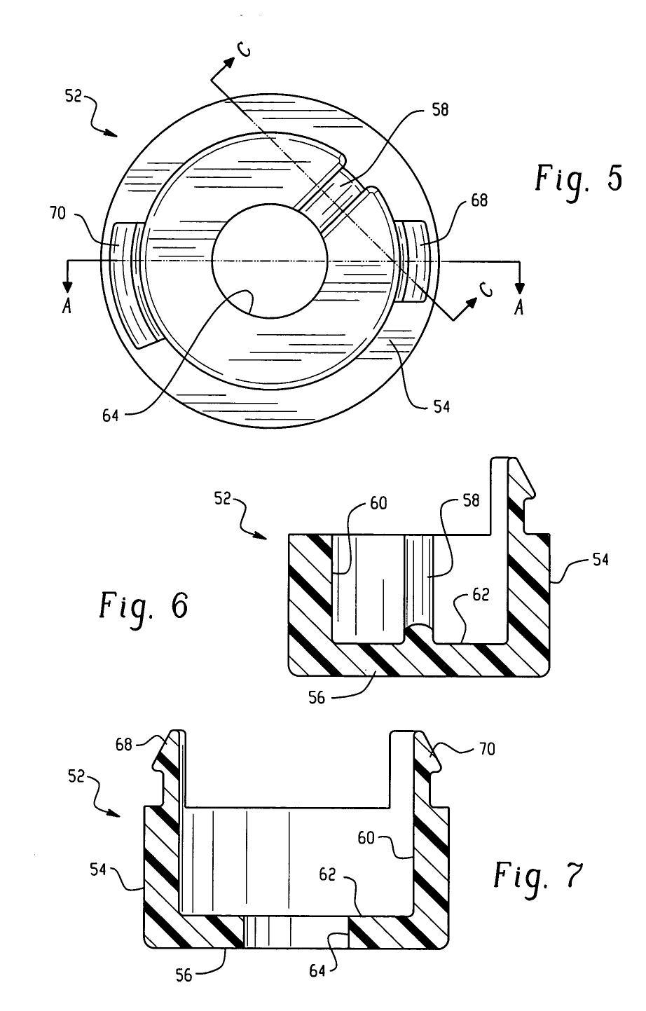

Referencing FIGS. 3-7, the bleed valve 44 comprises a hollowed body 52 comprising a circumferential wall 54 coupled to a radial wall 56. A stop 58 extends outward from an inner circumferential wall surface 60 and an inner radial wall surface 62. The radial wall 56 includes an orifice 64 therethrough that is generally opposite a larger orifice 66 defined by the circumferential wall 54 and two detents 68, 70 extending from the circumferential wall 54. The detents 68, 70 are adapted to be secured within two corresponding recesses 72, 74 (see FIG. 1) within the housing 28 to mount the bleed valve 44 thereto. The larger orifice 66 of the hollowed body 52 provides a throughput for insertion of a dual helix 76 adapted to be seated within the hollowed body 52.

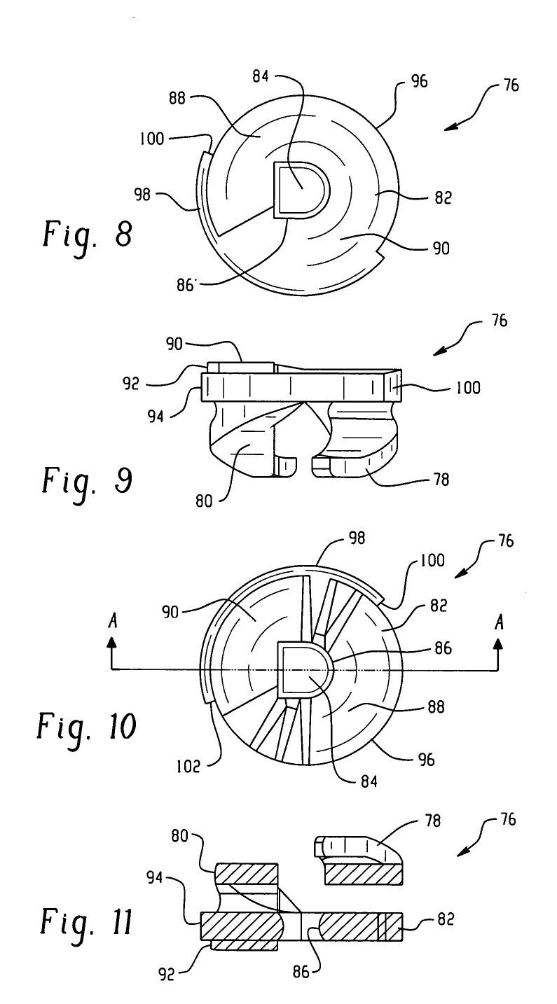

Referencing FIGS. 8-11, the dual helix 76 includes two spiral legs 78, 80 extending from a disc 82 having an axial D-shaped orifice 84 therethrough. The D-shaped orifice 84 includes a circumferential projection 86 that tapers in an hourglass shape. A top surface 88 of the disc 82 is radially planed to provide a cam surface 90 having a radial step 92. An outer circumferential surface 94 of the disc 82 is comprised of a first segment 96 and a second segment 98 divided by two circumferential steps 100, 102 evidencing the change in radial length between the segments 96, 98. The circumferential steps 100, 102 are adapted to interface with the stop 58 to inhibit rotation of the dual helix 76 within the hollowed body 52 beyond 210 degrees.

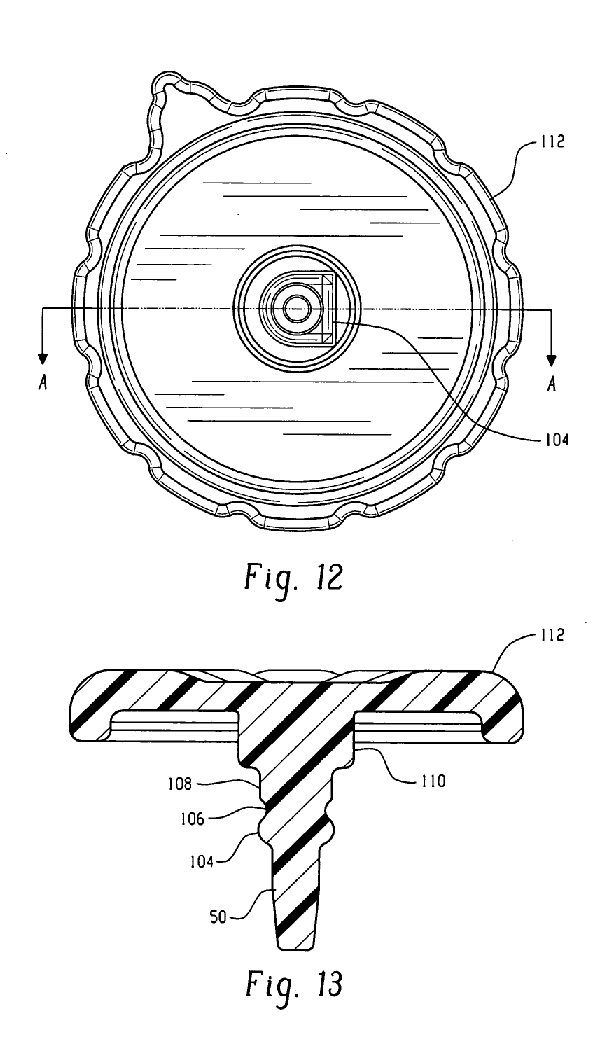

Referring to FIGS. 3, 4, 8, 12, and 13, the smaller orifice 60 of the hollow body 52 provides a throughput for receiving the needle 50 therethrough. The needle 50 includes a D-shaped key 104 riding circumferentially thereabout which is adapted to pass through the smaller orifice 60 irrespective of orientation. The D-shaped key 104 is also adapted to pass through the axial D-shaped orifice 84 centered within the disc 82 of the dual helix 76 upon proper orientation. The needle 50 includes a recess 106 adjacent to the D-shaped key 104 and a block 108 adjacent to the recess 106. The recess 106 is adapted to receive the circumferential projection 86 of the D-shaped orifice 84 to mount the dual helix 76 onto the needle 50. The needle 50 also includes a cylindrical segment 110 having a diameter slightly smaller than the diameter of the smaller orifice 60 to allow actuation of the needle 50 by a dial 112 coupled thereto.

Referring to FIGS. 3, 4, 8, 12, and 13, clockwise or counterclockwise rotation of the dial 112 repositions the needle 50 with respect to the opening 48 associated with the passage. The hollowed body 54 of the bleed valve 44 is secured to the housing 28 via the detents 68, 70, thereby allowing rotation of the needle 50 and dual helix 76 therein. The cam surface 90 rides upon the stop 58 to transform the rotation of the dial 112 into linear motion of the needle 50 with respect to the opening 48. For example, clockwise rotation of the dial 112 results in clockwise rotation of the needle 50, causing clockwise rotation of the dual helix 76 such that the stop 58 rides upon the cam surface 90 to increase the distance between the D-shaped key 104 of the needle 50 and the inner radial wall surface 62 of the radial wall 56 thereby moving the needle 50 closer to the opening 48, eventually plugging the opening 48 upon sufficient clockwise rotation. Conversely, counterclockwise rotation of the dial 112 results in moving the needle 50 farther from the opening 48 creating a correspondingly larger passage for fluid through the opening 48.

Referencing FIGS. 1 and 2, to withdraw milk from a breast, the liner 16 and funnel 12 are placed adjacent to the breast. A fluidic seal is created therebetween as the piston 30 is withdrawn from the front of the depressurized chamber 26 to increase the available volume for fluid occupying the funnel 12, diversion tunnel 14, vent 24, and depressurized chamber 26. This increase in available volume leads to a decrease in pressure that creates a pressure differential between fluid within the funnel 12, diversion tunnel 14, vent 24, and pressurized chamber 26 and the milk within the breast. Milk is secreted from the breast in accordance with fluid dynamics principles dictating fluid flow from high to low pressure areas. Those of ordinary skill are very familiar with such fluid dynamics principles.

The milk secreted from the breast is carried by the funnel 12 and into the diversion tunnel 14. Once in the diversion tunnel 14, the milk is gravity feds to the check valve 20. The check valve 20 comprises at least one flexible flap 114 allowing the milk to flow past the flap and enter the container 22. Upon depressurization (i.e., the piston 30 being withdrawn from the front of the depressurized chamber 26), the flap 114 seals off the contents of the container 22, thereby discontinuing fluid communication between the contents of the container 22 and the diversion tunnel 14. As milk enters the container 22, air is displaced and flows through the valve 20 and mildly increases the pressure within the system.

A high pressure differential between the depressurized chamber 26 and the breast may cause discomfort in the breast region. Such a pressure differential may be manipulated by using at least two methods. A first method includes slowing the withdrawal stroke of the piston 30 within the depressurized chamber 26. When the stroke is slowed, the milk withdrawn from the breast occupies a larger percentage of the volume within the breast pump 10 and is operative to decrease the pressure differential between the depressurized chamber 26 and the breast. However, as the piston 30 is biased by way of the leaf spring 34 coupled to the handle 32, this option may become tiresome.

A second method includes providing the bleed valve 44 as discussed above. By including the bleed valve 4, higher pressure fluid, typically air, is allowed to enter the depressurized chamber 26 by way of the opening 48 and passage to reduce the pressure differential between the depressurized chamber 26 and the breast. The bleed valve 44 may include various positions corresponding to unique flow rates for fluid to enter the depressurized chamber 26 from the atmospheric pressure environment and thereby reduce the pressure differential slowly or more quickly.

It should be understood that the above methods for manipulating the internal pressure within the breast pump 10 may be used in combination or exclusively.

Following from the above description and invention summaries, it should be apparent to those of ordinary skill in the art that, while the methods and apparatuses herein described constitute exemplary embodiments of the present invention, the inventions contained herein are not limited to these precise embodiments and that changes may be made to them without departing from the scope of the invention as defined by the claims. Additionally, it is to be understood that the invention is defined by the claims and it is not intended that any limitations or elements describing the exemplary embodiments set forth herein are to be incorporated into the meanings of the claims unless such limitations or elements are explicitly recited in the claims. Likewise, it is to be understood that it is not necessary to meet any or all of the identified advantages or objects of the invention disclosed herein in order to fall within the scope of any claim, since the invention is defined by the claims and since inherent and/or unforeseen advantages of the present invention may exist even though they may not have been explicitly discussed herein.

1. A pressure regulation device for selectively regulating the flow of a fluid between a lower pressure sink of a breast pump and a higher pressure source, the pressure regulation device comprising:
a valve body oriented with respect to a valve seat to selectively provide fluid communication between a lower pressure sink of a breast pump and a higher pressure source; and
an actuator operatively coupled to at least one of the valve body and the valve seat to manipulate the orientation of the valve seat with respect to the valve body;
wherein the actuator is repositionable to selectively manipulate a volumetric flow of fluid between the higher pressure source and the lower pressure sink by varying the proximity of the valve seat with respect to the valve body.

2. The pressure regulation device of claim 1, where in the valve body moves vertically with respect to the valve seat.

3. The pressure regulation device of claim 1, wherein the actuator is manipulated in one of a first plane and a second plane and the movement of the valve body with respect to the valve seat is in a third plane.

4. The pressure regulation device of claim 1, wherein the valve body includes a needle repositionable with respect to the valve seat.

5. The pressure regulation device of claim 1, wherein the actuator is coupled to the valve body and rotated coaxially with the valve body to manipulate the position of the valve body with respect to the valve seat.

6. A pressure regulation device for selectively regulating the flow of a fluid between a lower pressure sink of a breast pump and a higher pressure source, the pressure regulation device comprising:
a valve body oriented and biased with respect to a valve seat to selectively provide fluid communication between a lower pressure sink of a breast pump and a higher pressure source; and
an actuator operatively coupled to at least one of the valve body and the valve seat to manipulate the orientation of the valve seat with respect to the valve body;
wherein the actuator is repositionable to selectively manipulate a volumetric flow of fluid between the higher pressure source and the lower pressure sink by varying the bias of the valve body with respect to the valve seat.

7. The pressure regulation device of claim 6, wherein decreasing the bias increases the volumetric flow of fluid from the higher pressure source to the lower pressure sink.

8. The pressure regulation device of claim 6, wherein increasing the bias decreases the volumetric flow of fluid from the higher pressure source to the lower pressure sink.

9. The pressure regulation device of claim 6, wherein the lower pressure sink is developed at least in part by a piston traveling within a conduit in at least one of an arcuate path and a linear path.

10. The pressure regulation device of claim 9, wherein the piston is manually repositioned within the conduit by a user.

11. The pressure regulation device of claim 6, wherein:
the valve body is biased by a helical body coupled thereto;
the actuator is coupled to the valve body;
the valve body is co-axial with the helical body.

12. The pressure regulation device of claim 11, further comprising a breast pump body having a breast interface conduit adapted to create a fluidic seal between an outer circumferential area of the breast interface conduit and a breast, the breast body further including a lower pressure sink operative to draw milk through the breast interface conduit and into a milk reservoir.

13. The pressure regulation device of claim 12, wherein the breast pump body includes the valve seat.

14. The pressure regulation device of claim 12, wherein the actuator is rotated axially with respect to the valve body.

15. The pressure regulation device of claim 12, wherein the helical body, actuator, and at least a portion of the valve body are external to the breast pump body.

16. The pressure regulation device of claim 14, wherein rotation of the actuator is operative to increase or decrease the bias attributable to the helical body coupled to the valve body.

17. A pressure regulation device for selectively regulating the flow of a fluid between a lower pressure sink of a breast pump and a higher pressure source, the pressure regulation device comprising:
a valve body oriented with respect to a valve seat to selectively provide fluid communication between a lower pressure sink of a breast pump and a higher pressure source; and
an actuator operatively coupled to at least one of the valve body and the valve seat to manipulate the orientation of the valve seat with respect to the valve body;
wherein the actuator transforms rotational movement into linear movement of at least one of the valve body and the valve seat to reposition the valve body with respect to the valve seat.

18. A breast pump comprising:
an interface adapted to create a fluidic seal between a circumferential portion of the interface and a breast;
a reservoir in fluid communication with the interface for receiving milk drawn from the breast and passing by the interface;
a sink in fluid communication with the interface, where the sink induces a reduced pressure approximate the interface to draw milk from the breast and past the interface and into the reservoir; and
a pressure regulator in fluid communication with the sink to regulate the reduced pressure approximate the interface;
wherein the pressure regulator includes a valve seat and a valve body that are selectively repositionable to manipulate the reduced pressure approximate the interface by varying the proximity of the valve seat with respect to the valve body.

19. The breast pump of claim 18, wherein the sink includes a chamber comprising a conduit having a piston riding therein, the piston being repositionable within the conduit to induce the reduced pressure approximate the interface.

20. The breast pump of claim 19, wherein the piston travels within the conduit in at least one of an arcuate path and a liner path.

21. The breast pump of claim 18, wherein the pressure regulator includes a dial actuator being repositionable in at least one of a clockwise direction and a counterclockwise direction to vary the proximity of the valve seat with respect to the valve body.

22. The breast pump of claim 19, wherein the piston is coupled to a handle being repositionable by a user.

23. A pressure regulation device for selectively regulating the flow of a fluid between a lower pressure sink within a breast pump and a higher pressure source, the pressure regulation device comprising:
a valve plug circumferentially bounded, at least in part, by a housing adapted to contact a helical body to bias the valve plug with respect to a valve seat where the position of the helical body with respect to the housing determines at least in part the position of the valve plug with respect to the valve seat; and
an actuator operatively coupled to the helical body to manipulate the position of the valve plug with respect to the valve seat, thereby selectively providing fluid communication between a lower pressure sink of a breast pump and a higher pressure source.

24. The pressure regulation device of claim 23, wherein the helical body includes a helical cam surface adapted contact the housing operative to manipulate the position of the valve plug with respect to the valve seat.

25. The pressure regulation device of claim 24, wherein the actuator is rotatably actuated.

26. The pressure regulation device of claim 23, wherein combination of the actuator, the helical body, and the housing transform rotational movement into linear movement of the valve plug with respect to the valve seat.

27. The pressure regulation device of claim 26, wherein the housing and helical body each accommodate vertical throughput of the actuator.

28. The pressure regulation device of claim 27, wherein the helical body is inhibited from rotation adjacent to the housing of greater than 360 degrees.

29. The pressure regulation device of claim 27, wherein the valve plug is repositionable with respect to the valve seat to accommodate a plurality of volumetric flow rates of fluid between the higher pressure source and the lower pressure sink.

30. A pressure regulation device for selectively regulating the flow of a fluid between a lower pressure sink within a breast pump and a higher pressure source, the pressure regulation device comprising a dial actuator including an appendage coupled to a valve body, at least one of the appendage and the valve body having a helical body mounted thereto, the helical body biasing the dial actuator, where rotation of the dial actuator varies the position of the valve body with respect to a valve seat, having an orifice therein, for selectively regulating a volumetric flow of a fluid between a lower pressure sink within a breast pump and a higher pressure source.

31. The pressure regulation device of claim 30, further comprising a housing adapted to be mounted to the breast pump, the housing includes a cavity occupied at least in part by the helical body, where an adjacent surface of the housing interfaces with the helical body such that rotation of the helical body with respect the adjacent surface varies the position of the valve body with respect to a valve seat.

32. The pressure regulation device of claim 31, wherein the housing is a cylindrical body having at least one fastener adapted to interface with the breast pump to attach the housing thereto, wherein the adjacent surface includes at least one protrusion adapted to contact the helical body.

33. The pressure regulation device of claim 32, wherein the helical body includes a disc with a contoured surface adapted to interface with the adjacent surface of the housing, the contoured surface having an opening therethrough for allowing throughput of at least one of the appendage and the valve body, the disc further including at least two helical appurtenances opposite the contoured surface that are adapted to interface with the breast pump.
